(19) United States
(12) Patent Application Publication
(10) Pub. No.: US 2026/0232193 A1
(43) Pub. Date: Aug. 13, 2026

(54) IMAGING METHOD, APPARATUS AND ASSOCIATED COMPUTER PROGRAM PRODUCT

(71) Applicants: UNIVERSITE DE LIMOGES, Limoges Cedex (FR); CENTRE NATIONAL DE LA RECHERCHE SCIENTIFIQUE, Paris 16 (FR)

(72) Inventors: Mohammad OJAROUDI PARCHIN, Limoges (FR); Stéphane BILA, Verneuil-sur-Vienne (FR)

(21) Appl. No.: 19/149,311

(22) PCT Filed: Dec. 22, 2023
(86) PCT No.: PCT/IB2023/063178

§ 371 Date: Jul. 18, 2025

(30) Foreign Application Priority Data
Jan. 18, 2023 (FR) FR2300441

(51) Int. Cl. A61B 5/00; A61B 5/0507

(52) U.S. Cl. CPC A61B 5/0042; A61B 5/0507; A61B 5/4064; A61B 5/7271; A61B 2576/026

(57) ABSTRACT

The present invention relates to an imaging method, an imaging apparatus, and an associated computer program product for creating a three-dimensional image by microwave imaging of the brain of a patient using target detection in the brain to reduce the imaged region in subsequent scans in order to enable real-time monitoring of the history of the target in the brain of the patient.

### TECHNICAL FIELD

The present invention relates to the technical field of medical imaging, and more particularly to an imaging method, an apparatus, and an associated computer program product.

Brain diseases are the subject of intense research, both in terms of their treatment and diagnosis.

Once diagnosed, certain brain conditions require monitoring to prevent more severe states in patients. Thus, clinical study results suggest that in patients suffering from primary intracerebral hemorrhage, the risk of rebleeding is not negligible: 24% of patients experienced one or more rebleeding episodes during an average follow-up period of 84.1 months. This risk appears to be highest during the first year following the initial hemorrhage. The frequency of a second hemorrhage was higher in those whose blood pressure was not controlled, according to these same studies. In fact, the higher the blood pressure of the patient, the greater the risk of recurrence.

It is therefore important to find methods for monitoring the condition of the patient, either on a periodic model or on a one-time model for patients for whom an imminent stroke risk has been determined. These methods can be anatomical, biological, physiological, but essentially rely on brain imaging, with the problem that, while it can be effective, brain imaging requires expensive and difficult to transport equipment, and requires calculations that are not easily compatible with rapid diagnosis.

In approaches focusing on monitoring a condition, such as the evolution of a stroke, Alzheimer's disease, epilepsy, Parkinson's disease (PD), and Tourette's syndrome, the signals must be based on anatomical and physiological information extracted during tissue scanning and be selected comparatively to improve the speed of extraction of the images produced in terms of concentration on changes in position and structure of the target. Current imaging methods, such as magnetic resonance imaging (MRI) and computed tomography (CT), are thus difficult functional MRI (EMRI) to apply. Moreover, and electroencephalogram (EEG), which are currently the most used techniques as they can provide excellent detailed anatomical and physiological information related to the monitoring of brain disorders, have drawbacks: the temporal resolution of conventional fMRI is rather low and takes a few seconds to reach its peak, while its spatial resolution of around a square millimeter is excellent, whereas EEG has a temporal resolution of around a millisecond, but its spatial resolution is rather low and covers a few square centimeters.

In support of these imaging methods, computational brain imaging has been one of the most extensive and challenging research areas in recent years. Due to the complex nature of brain diseases, various approaches have been explored over the past decades, each with its advantages and disadvantages. Brain microwave imaging based on the electromagnetic approach has attracted considerable attention in recent years due to its advantages, such as its non-invasive nature, lack of contact, low cost, and portability. Extracting information about the brain in these systems still presents many challenges. Various processing methods have been proposed to increase the capacity of these devices, as well as the usefulness of the extracted images to detect and diagnose brain diseases, including strokes. These conventional approaches require a large number of antennas to collect the scattered inverse field and thus solve the inverse scattering problem, resulting in high processing time.

The invention aims to enable continuous monitoring of strokes, applied to monitoring bleeding and blood clot proliferation in stroke patients, using an emerging brain imaging system based on an electromagnetic approach, and allows for an ultra-fast and super-resolution monitoring methodology. In this regard, unlike other conventional static microwave imaging systems that can be considered as performing blind scanning, the dynamic microwave imaging system based on a multi-static network according to the invention has great potential to provide useful tools for stroke monitoring. The main objective is to develop ultra-fast destructive resolution methods to pave the way for cerebrovascular monitoring by performing closed-loop cognitive scans based on a common algorithm/design.

The hypothesis is that, unlike differential imaging where time-varying activities are detected by subtracting static closed-loop functional imaging is information, cognitive modulated in real-time from by feedback recordings of intelligent irradiation backscatter that is faster and can extract more information. To do this, according to the dynamic changes in brain activities, the transmitted waveforms, and thus the activated structures of the sub-networks and focus networks, will be cognitively modified and continuously adapted to the changing targets. The radical vision proposed, based on cognitive scanning (CS), uses an information-based approach for the tested scenario. The phenomenon behind this idea is that, for monitoring and functional imaging, it is not necessary to detect the entire volume of the brain, but only to save/use the data that contains relevant information about a predefined set of brain activities. To transform the problem into an informational problem, it is necessary to obtain the basic parameters of knowledge-based information synthesis, one of which is the existence of prior knowledge or a library to create a knowledge development system and online decision autonomy. The main argument for the superiority of cognitive scanning for functional brain imaging is that it is more flexible for unknown patterns of brain activities than multi-static imaging and can therefore be more widely proposed as a monitoring option with fewer contraindications. By identifying an activated region as a target of interest, the microwave imaging scenario is transformed into a microwave detection scenario.

The present invention therefore relates to a method of three-dimensional brain imaging by microwave imaging, characterized by comprising the following steps:
a—performing, on a patient on whose head is placed a microwave antenna array controlled by a switching network connected to a controllable signal transceiver, a global three-dimensional image of the brain by microwave imaging using all the antennas of the microwave antenna array;
b—detecting at least one target in the global brain image made in step a;
c—calculating a position of the at least one target in the global brain image made in step a;
d—performing a local three-dimensional processing image of the target by microwave imaging using at least one among a subgroup of the microwave antenna array controlled by the switching network associated with the position of the at least one target detected in step a and a reduction in the number of samples made by the antennas;
e—merging the global image and the local processing image into a global intermediate three-dimensional image;
f—detecting least one target in the global intermediate image;
g—calculating a position of the at least one target in the global intermediate image;
h—performing a new local three-dimensional processing image of the target by microwave imaging using at least one among a subgroup of the microwave antenna array controlled by the switching network associated with the position of the at least one target detected in step f and a reduction in the number of samples made by the antennas;
i—merging the global intermediate image and the new local processing image into a new global three-dimensional image also called intermediate image;
j—repeating steps f to i a predetermined number of times.

Advantageously, the antenna array is formed on a helmet, the antennas being evenly distributed on the helmet in a hemispherical manner. Advantageously, the array comprises 24 antennas, although the invention is not limited in this regard.

The antennas are transmitting and receiving antennas, allowing microwave signals to be transmitted and received.

Thus, by means of the invention, due to the reduced number of antennas and/or samples when making a local image due to the localization of the target, fewer calculations are needed and thus the image is obtained more quickly, allowing more real-time monitoring. The global image not being recalculated at each iteration, we are thus only interested in the monitoring and imaging of the target, which reduces the acquisition and calculation of the image. However, at each iteration, a target is searched for in the global image, which ensures that no target is missed, particularly in the context of functional imaging where we want to understand the evolution of the size, location, and nature of the target depending on the condition of the patient, even if the entire image is not recalculated at each iteration. The fact that the system comprises all the antennas, but that they are only activated according to the location of the target allows great flexibility, reduced computational complexity, and high target detection efficiency. In addition, less powerful computing equipment is required compared to the state of the art. Finally, the image can be taken by a simple helmet equipped with microwave antennas connected to a computing device, allowing portability that is not possible in current methods and systems.

Advantageously, for better efficiency, both a subgroup of the antenna array and a reduction in the number of samples are used, which further reduces the computational load.

The invention also relates to a brain microwave imaging apparatus, characterized by comprising a helmet equipped with a microwave antenna array, a switching network controlling the microwave antenna array, a vector network analyzer connected to the switching network, a processing unit connected to the switching network and the vector network analyzer and controlling the switching network and the vector network analyzer, a human-machine interface connected to the processing unit, the processing unit comprising computing means and memory to perform the following steps:
a—performing a global three-dimensional image of the brain by microwave imaging using all the antennas of the microwave antenna array;
b—detecting at least one target in the global brain image made in step a;
c—calculating a position of the at least one target in the global brain image made in step a;
d—performing a local three-dimensional processing image of the target by microwave imaging using at least one among a subgroup of the microwave antenna array controlled by the switching network associated with the position of the at least one target detected in step a and a reduction in the number of samples made by the antennas;
e—merging the global image and the local processing image into a global intermediate three-dimensional image;
f—detecting at least one target in the global intermediate image;
g—calculating a position of the at least one target in the global intermediate image;
h—performing a new local three-dimensional processing image of the target by microwave imaging using at least one among a subgroup of the microwave antenna array controlled by the switching network associated with the position of the at least one target detected in step f and a reduction in the number of samples made by the antennas;
i—merging the global intermediate image and the new local processing image into a new global three-dimensional image also called intermediate image;
j—repeating steps f to i a predetermined number of times.

The antennas are preferably arranged on the helmet in a hemispherical manner, distributed uniformly.

The processing unit allows signals to be transmitted to the helmet and signals from the helmet to be received.

According to one embodiment, the antenna array comprises 24 antennas, preferably butterfly antennas.

Advantageously, the switching network is a 2*24 switching matrix wherein the 24 ports are connected to the antennas and the two ports are connected to the vector network analyzer.

The invention also relates to a computer program product comprising instructions which, when executed by a computer, perform the following steps:
a—performing a global three-dimensional image of the brain by microwave imaging using all the antennas of the microwave antenna array;
b—detecting at least one target in the global brain image made in step a;
c—calculating a position of the at least one target in the global brain image made in step a;
d—performing a local three-dimensional processing image of the target by microwave imaging using at least one among a subgroup of the microwave antenna array controlled by the switching network associated with the position of the at least one target detected in step a and a reduction in the number of samples made by the antennas;
e—merging the global image and the local processing image into a global intermediate three-dimensional image;
f—detecting t least one target in the global intermediate image;
g—calculating a position of the at least one target in the global intermediate image;
h—performing a new local three-dimensional processing image of the target by microwave imaging using at least one among a subgroup of the microwave antenna array controlled by the switching network associated with the position of the at least one target detected in step f and a reduction in the number of samples made by the antennas;
i—merging the global intermediate image and the new local processing image into a new global three-dimensional image also called intermediate image;
j—repeating steps f to i a predetermined number of times.

According to one embodiment, each step of performing a three-dimensional image by microwave imaging comprises collecting diffusion parameter data representing microwaves scattered by the brain of the patient in a diffusion tensor, generating differential diffusion parameter data by an adaptive connectome calibration process to suppress clutter, and processing the differential diffusion parameter data by a confocal image reconstruction process to obtain a three-dimensional image.

The adaptive connectome calibration process involves calculating the geometric connectome between the hemispherical antenna array and the signals passing through the brain of the patient, the term connectome being defined in the present application as the set of geometric connections/links between the antennas and the signals. Depending on the symmetry of the medium traversed by the microwave signals, it is possible to categorize the signals by differential pairs that traverse symmetrical paths. By performing a subtraction between these symmetrical signals, all information unrelated to the target is removed. In practice, the signals are regrouped using an intercorrelation matrix, wherein the signals from channels that follow the same path through the brain of the patient and have a high correlation value are classified into identical groups. Differential diffusion parameter data is obtained by subtracting the signals from each group from each other. In subsequent scans, where the signals whose differential pair has been removed due to the abandonment of a certain number of antennas in the antenna arrangement optimization process, an adaptive mode is used to determine the differential pair: the correlation of each signal is calculated with the rest of the signals, and the signal that gives the highest correlation value is selected as the differential pair.

The confocal image reconstruction process uses a delay-multiply-and-sum (DMAS) beamforming by processing the differential diffusion parameter data. The maximum value of the coherently focused energy in the reconstructed image refers to the functional brain region of interest, for example, a stroke region. The location of these areas is considered the position of a possible target. Such a technique is described, for example, in the following publications:
Ahadi, M., Isa, M., Saripan, M. I. and Hasan, W. Z. W. (2015), Three dimensions localization of tumors in confocal microwave imaging for breast cancer detection, Microw. Opt. Technol. Lett., 57:2917-2929;
Salvador, Sara M., and Giuseppe Vecchi, Experimental tests of microwave breast cancer detection on phantoms, IEEE Transactions on Antennas and Propagation 57, no. 6 (2009): 1705-1712;
Babarinde, O. J., Jamlos, M. F., Soh, P. J., Schreurs, D. P. and Beyer, A., Microwave imaging technique for lung tumour detection, 2016 German Microwave Conference (GeMiC), 2016, pp. 100-103.

According to one embodiment, the at least one target is detected by calculating the signal-to-clutter ratio (SCR) and the signal-to-mean ratio (SMR) in two-dimensional planes of the three-dimensional image, the at least one target being detected on the surfaces of each two-dimensional plane on which the values of the signal-to-clutter ratio and the signal-to-mean ratio are simultaneously maximal. Such a technique is described, for example, in the following publications:
Reimer, T., Solis-Nepote, M. and Pistorius, S., 2020, The application of an iterative structure to the delay-and-sum and the delay-multiply-and-sum beamformers in breast microwave imaging, Diagnostics, 17/06/2022, 10(6), p. 411;
Babarinde, O. J., Jamlos, M. F., Soh, P. J., Schreurs, D. P. and Beyer, A., Microwave imaging technique for lung tumour detection, 2016 German Microwave Conference (GeMiC), 2016, pp. 100-103.

According to one embodiment, the position of the at least one target in the three-dimensional image is calculated as the position of the surfaces of each two-dimensional plane on which the values of the signal-to-clutter ratio and the signal-to-mean ratio are simultaneously maximal. The probability that the target is detected is thus maximal.

Thus, the location of the target region is approximated by superimposing quantitative metric maps, such as the maximum value for the signal-to-clutter ratio (SCR) and the signal-to-mean ratio (SMR) in a two-dimensional plane, and the spatial dimensions of the target region correspond to the selected SCR and SMR windows.

According to one embodiment, the subgroup of the microwave antenna array associated with the position of the at least one target is constituted by the antennas having the shortest distance to the position of the at least one target.

According to one embodiment, compressive sampling is applied to the microwave signals emitted by the antennas of the antenna array. The computing power to be implemented is thus reduced. Compressive sampling is advantageously implemented by a convex optimization method of the L¹ space (which in the present application designates the space of functions with values in [symbol] whose absolute value (or the space of functions with values in [symbol] whose module) is integrable in the sense of Lebesgue).

According to one embodiment, each step of merging two three-dimensional images consists of replacing, in the global image, the part corresponding to the local image, with the local image to obtain the merged image.

Since the position of the target is known, the region of the global image corresponding to the target can be known, and this region in the global image can be replaced by the local image of the target.

Only the additional information obtained during processing is added to the global 3D image.

The predetermined number of times the steps f to i are repeated depends on the choice of the operator using the device according to the invention. The predetermined number of times can in particular be correlated to a duration, in particular a duration of patient monitoring, or be correlated to an absence of evolution of a target over time (imaging by the device is stopped if the target no longer evolves after a certain duration) or to too rapid evolution of the target (significant spatial development of the target over a predetermined duration).

After step j, the obtained image is an image of the brain of the patient on which the target is positioned in space. An evolution of the target over time is also obtained, from step a to step j by storing and comparing the images.

The invention therefore uses an intelligent cognitive scanning program. The imaging device of the invention uses a dynamic intelligent structure to create high-resolution images in the shortest possible time. The cognitive scanning paradigm proposed in this invention is the result of a combination of information theory and electromagnetic theory. In multi-scan dynamic mode, feedback is used to increase input data for subsequent scans. The selection of the antenna array arrangement that determines the desired signals in the processing step depends on the information from the previous scan and is selected to extract new information in the next scan. The brain is irradiated by an antenna array that propagates the electromagnetic wave into the head. These waves are then converted into measured reflected signals. After passing through the receiver, they are converted into data depending on the type of scan. From this path, information is extracted from the captured data.

Unlike conventional multi-static microwave imaging systems, the proposed cognitive scanning also provides an additional information extraction/integration process. The main objective presented here is to create a closed-loop cognitive scanning as an alternative to differential imaging that is faster and allows more information to be extracted. Based on compression measurement and cognitive sampling techniques, both the information flow and the results of the proposed device create highly innovative decision-making schemes for ultra-fast dynamic microwave imaging systems. Based on this capability, the simultaneous design of a multi-scan algorithm architecture based on cognitive scanning, which comprises antenna selection and sub-Nyquist sampling, is implemented. Compression-based sampling allows ultra-fast sampling and complete reconstruction of certain classes of unlimited signals. In information-based scanning, temporal and spatial changes in the monitoring scenario are encountered. These temporal and spatial changes must be intelligently detected and tracked. In addition, only variant samples must be extracted by scanning. The extracted data also adds new information to the knowledge growing system (KGS) library to estimate the future state of the target of interest (TOI). To this end, the invention combines algorithms with architectures for cognitive scanning, data acquisition, and integration of the information required for very fast tracking to optimize performance.

The main argument for the superiority of cognitive scanning for static brain imaging is that it is faster and more accurate for multi-static brain imaging and can therefore be more widely proposed as a monitoring option with fewer contraindications. By identifying the target area as a target of interest, the microwave imaging scenario is transformed into a detection scenario.

To better illustrate the object of the present invention, a preferred embodiment will be described below, in connection with the attached drawings.

In these drawings:

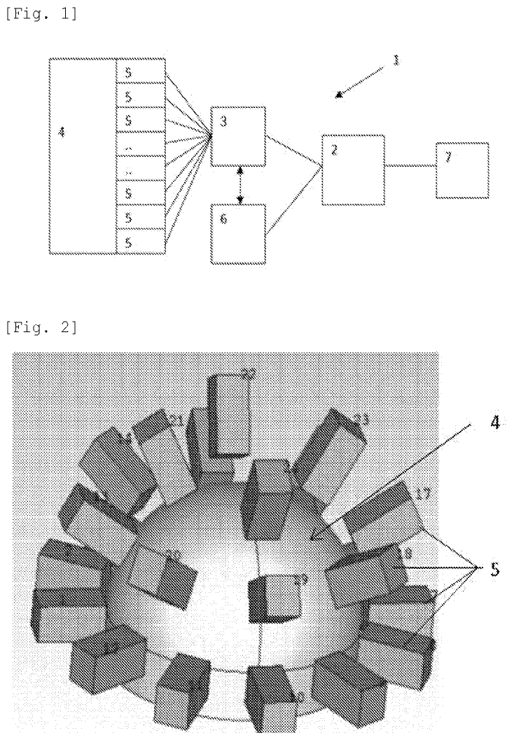

FIG. 1 is a block diagram of the microwave imaging device according to the present invention;

FIG. 2 is an exemplary representation of a helmet used by the device from FIG. 1;

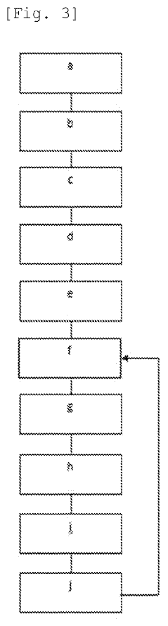

FIG. 3 is a flowchart of the imaging process implemented by the microwave imaging device according to the present invention;

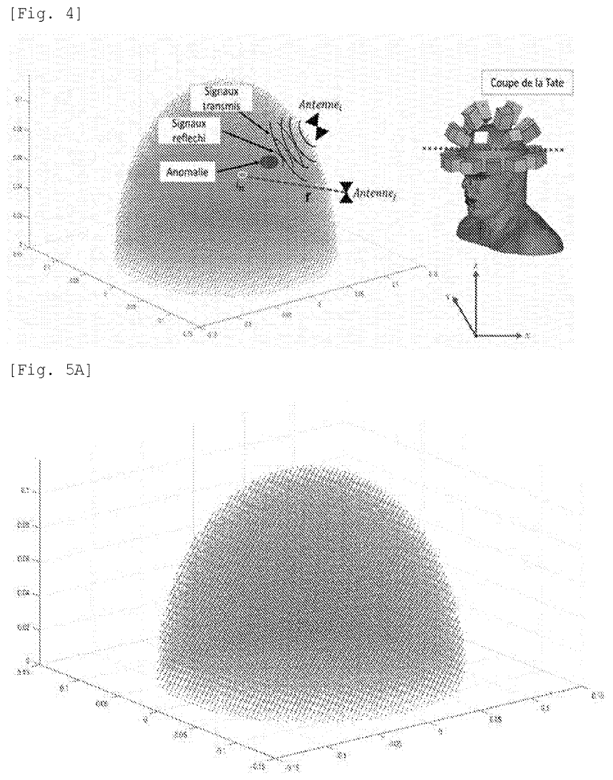

FIG. 4 represents a two-dimensional imaging domain of the human head;

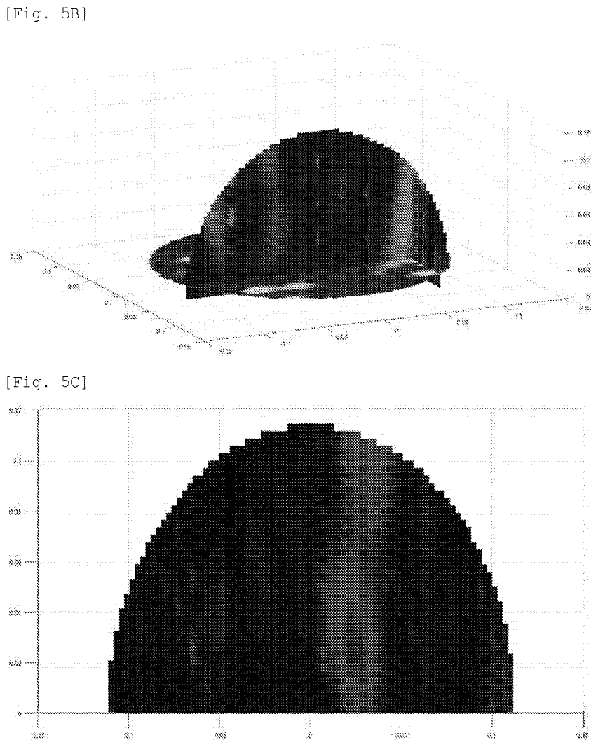

FIG. 5A represents a first step of image reconstruction by a confocal image reconstruction process;

FIG. 5B represents a second step of image reconstruction by a confocal image reconstruction process;

FIG. 5C represents a third step of image reconstruction by a confocal image reconstruction process;

FIG. 5D represents a fourth step of image reconstruction by a confocal image reconstruction process;

FIG. 6 represents a 2D image extracted according to the invention; and

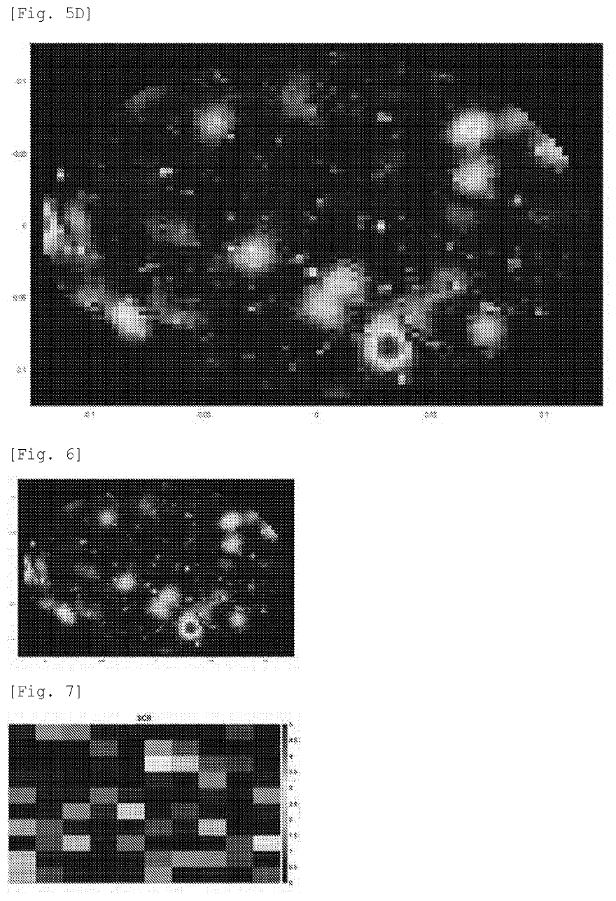

FIG. 7 represents the 2D image obtained from the image in FIG. 6 based on the SCR metric.

Such a method is described, for example, in the publication O'Loughlin, D., Elahi, M. A., Lavoie, B. R., Fear, E. C. and O'Halloran, M., 2021, Assessing Patient-Specific Microwave Breast Imaging in Clinical Case Studies, Sensors, 21(23), p. 8048.

Referring to FIG. 1, it can be seen that a microwave imaging device 1 according to the present invention is schematically represented.

The device 1 comprises a processing unit 2, comprising computing means, such as a microprocessor, microcontroller, digital signal processor (DSP), processor, field-programmable gate array (FPGA), or application-specific integrated circuit (ASIC), associated with memory (such as ROM, EEPROM, RAM, flash memory), as well as input/output ports and/or wired or wireless communication means with the other elements of the device 1, to implement the steps described below for microwave brain imaging and transmit signals to the other elements of the device 1 and receive signals from the other elements of the device 1.

The device 1 further comprises a switching network 3 connected to a helmet 4 carrying a plurality of antennas 5, the antennas 5 being controlled by the switching network 3, which also collects signals from the antennas 5, a vector network analyzer 6, connected to the switching network 3 and processing the signals from the switching network 3 to send them to the processing unit 2, and a human-machine interface 7, comprising a screen and input means, such as a keyboard and/or mouse.

The processing unit 2 and the human-machine interface 7 can, for example, be implemented by means of a conventional desktop computer or a tablet or even a smartphone and is configured to implement the steps described below.

Referring to FIG. 2, it can be seen that a helmet 4 equipped with its antennas 5 is represented according to a preferred embodiment.

The helmet 4 is hemispherical in shape, the antennas 5 being 24 in number in this exemplary and non-limiting embodiment, distributed uniformly on the surface of the helmet 4. The numbers on the antennas are used to reference the antennas 5 for control. The number assigned to each antenna 5 in FIG. 2 is of course illustrative and non-limiting. The invention is not limited to the number of 24 antennas indicated in this exemplary and non-limiting implementation of the invention.

The antennas 5 are preferably butterfly antennas and advantageously have a bandwidth of about 3 GHZ.

The processing unit 2 is configured in the microwave imaging device 1 to implement the following steps, schematically represented in FIG. 3, when the helmet 4 is placed on the head of a patient:
a—performing a global three-dimensional image of the brain by microwave imaging using all the antennas 5 of the microwave antenna array 4;
b—detecting at least one target in the global brain image made in step a;
c—calculating a position of the at least one target in the global brain image made in step a;
d—performing a local three-dimensional processing image of the target by microwave imaging using at least one among a subgroup of the microwave antenna array 4 controlled by the switching network 3 associated with the position of the at least one target detected in step a and a reduction in the number of samples made by the antennas 5;
e—merging the global image and the local processing image into a global intermediate three-dimensional image;
f—detecting at least one target in the global intermediate image;
g—calculating a position of the at least one target in the global intermediate image;
h—performing a new local three-dimensional processing image of the target by microwave imaging using at least one among a subgroup of the microwave antenna array 4 controlled by the switching network 3 associated with the position of the at least one target detected in step f and a reduction in the number of samples made by the antennas 5;
i—merging the global intermediate image and the new local processing image into a new global three-dimensional image also called intermediate image;
j—repeating steps f to i a predetermined number of times.

After step j, the obtained image is an image of the brain of the patient on which the target is positioned in space. An evolution of the target over time is also obtained, from step a to step j.

The SCR and SMR metrics described below are used to detect the target in steps b and f.

The goal is to increase the speed and accuracy of image reconstruction by reducing hardware complexity to a minimum, provided that most of the information can be extracted in the software of the processing unit 2. In the present invention, a complete brain scan is performed and, at each scan, only the necessary samples are selected to reduce processing speed in the software of the processing unit 2.

A complete scan of the brain of the patient is therefore first performed.

A diffusion tensor related to the complete scan is created in the processing unit 2, the tensor being a matrix representing the signals transmitted and received by each antenna 5 of the helmet 4.

By applying the adaptive connectome calibration process detailed below to the diffusion tensor, the antennas 5 necessary for the scan are determined based on information about the target area.

A reduction in the number of samples (compressive sampling) is applied to the result of the adaptive connectome calibration process applied to the diffusion tensor to obtain a sparse matrix from the diffusion tensor. An improved sparse matrix is then created from the complete scan.

Compressive sampling is advantageously implemented on the improved sparse matrix by a convex optimization method of the L1 space.

To generate sparse signals, suppose the length of a signal x is M×1. If the signal is sparse with factor K (K<<M), it can be represented as x=ψs, where ψ is a complete orthogonal dictionary matrix. The dictionary matrix is used as a coefficient in the compressive sensing method. Compressive sensing, which is considered an inverse reconstruction of the original signal from sparse signals, uses the linear operation of the measurement matrix (φ) and the input signal as follows: y=φx. This theory is based on the assumption that the measurement matrix Φ is inconsistent with the dictionary matrix ψ. By this condition, the matrix vector, s, can be reconstructed from G=O(K*log Nt). To solve this equation, we need to solve the following convex optimization problem.

$$min 〚 ‖ s ‖ \_ {1}_{1} subject to y = ϕ ψ s 〛 \qquad [ Math . 1 ]$$

In the present invention, a convex optimization based on the L1 norm is proposed to solve this equation. In addition, matrix operations in compressive detection can be written as: y=Øψs=As, where Ø is a random Gaussian measurement matrix and ψ is the discrete cosine transform (DCT) matrix: A=Øψ. The compressive method is first applied to generate sparse signals. Then, from these sparse signals, images are obtained by the confocal image reconstruction process to compare the new SCR. If the SCR is still high, it means that the sparse signal is correct. Otherwise, the sparsity factor must be decreased.

Optimal sparsity is applied so that fewer non-zero elements are used in the range where there is no signal change. In general, from a temporal point of view, the initial part of the signal is often omitted because it has high values due to surface reflection. However, useful information can be extracted from these signals to be used in estimating certain parameters, such as effective permittivity, etc.

After collecting the received signals, all signals are reduced in size using sparse signals. Since most natural signals in one or more domains (time, frequency, wavelets, etc.) have a sparse display, it means that the information they contain can be used using a small number of coefficients in a special domain expressed. For example, although images appear very dense or rich in space and with a lot of information, in the frequency domain, they have compact and so-called thin information. Thus, most of their frequency coefficients are zero or close to zero. The same goes for many other types of signals. Therefore, a very large signal of length N can only be represented by coefficient K, which is K<<M; this signal is called a sparse K order.

From the sparse matrix, an image is created using the confocal image reconstruction process.

Meanwhile, to extract anatomical information, existing images related to the scan of the head of the patient, such as CT and/or MRI, are transmitted to the device 1, preferably wirelessly.

These existing images are then processed and segmented by a machine learning device to process geometric and position information of the target in the brain image.

The existing images are then merged with the image created by the confocal image reconstruction process, and a complete brain image is created.

This scenario being intended for brain monitoring, it has a multi-scan mode allowing target evolutions/changes to be revealed.

In radar imaging, after receiving the return signals using a multi-static scanning data collection method, a diffusion tensor is created. This step, which is formed from hardware to software, converts electromagnetic waves into complex data recorded in a tensor called a diffusion tensor. In this tensor, which is a matrix, depending on the useful information, the part below the diagonal is first eliminated, as well as the diagonal elements, which correspond to the signals reflected by each antenna, due to the much higher values than the other signals. In general, before converting signals into data and data into information, calibration must be performed in the hardware to ensure the accuracy of the received signals. Calibration comprises, in particular, eliminating couplings between antennas by adjusting their distance, creating a plane wave instead of a spherical wave by adjusting the distance of the measurement medium, and eliminating unwanted reflections by adding an absorber or a metal backplate to suppress scattering behind the antenna. Once all measures have been taken to transfer the correct data from hardware to software, the latter is calibrated.

Due to the reciprocity property of electromagnetic theory, it is not necessary to scan the entire 24 antennas×24 antennas array. In other words, the information stored from the 3D simulation model comprises the diagonal elements and the upper triangular elements of the diffusion tensor. The diagonal elements are the antenna return losses. The other signals are the transmission losses between different antenna pairs.

The main objective of the software part of the processing unit 2 is to reconstruct the precise image of the received signals. First, to map the information, a hemisphere of coordinates containing the focal points must be created. To create this hemisphere, physical information, such as the ambient radius, ambient material, and the number of sampling points must be entered into the program of the processing unit 2 via the human-machine interface 7. The ambient radius determines the image limit. The number of points can also be determined based on the signal bandwidth. Another important parameter is the environmental material, which is defined based on dielectric permittivity and electrical conductivity. Dielectric permittivity is more important due to wave speed changes. In the next step of entering parameters into the program, the information of the device 1 is entered, such as the number of antennas 5, their location, and the channels related to the signals, which show the relationship between the signals and the path between two respective antennas 5 in each signal.

After entering all the physical parameters, the first pre-processing section comprises the clutter suppression algorithm with in-situ calibration. In brain radar imaging, since there are several unknown parameters to reconstruct the global image, it must be extracted from the return signals. Thus, information theory-based methods can be very useful, both to increase processing speed and to detect certain targets by the device. In this invention, we propose the adaptive form of the in-situ calibration algorithm which is based on the dynamic arrangement of the connectome (set of connections between antennas and signals) in the feedback processing path. In this method, signals are selected in the diffusion tensor based on geometric information of the imaged medium (brain) and wave propagation channels in the environment. To this end, we perform treatments from different paths that lead more information being extracted. In general, all information is extracted from the diffusion tensor in addition to the physical information of the system to reconstruct the image.

The in-situ adaptive connectome calibration process uses the symmetrical property of the right and left sides of the elliptical structure of the brain.

All connections between antennas are identified.

The connections are then grouped by distance between a considered antenna pair. Thus, a first group is constituted by the connections between adjacent antennas, a second group is constituted by antennas separated by one antenna, a third group is constituted by antennas separated by two antennas, etc., the last group being constituted by antennas separated by the maximum number of possible antennas 5 on the helmet 4. These groups represent signals that travel symmetrical paths.

For the helmet 4 represented in FIG. 2, the last group would therefore be constituted by antennas 5 separated by five antennas (for example, the antennas 5 diametrically opposed on the helmet 4).

For each group of signals, the first signal is subtracted from the next, and the obtained signal is subtracted from the next until the last signal of the group. Thus, for the group of signals, a differential signal is obtained whose characteristics unrelated to the target to be detected are removed.

The differential signal obtained for a group is then subtracted from the average value of the signal for the group. These two steps allow clutter to be suppressed, notably the background and skin effects, to retain only the useful information related to the target.

Once this processing is done, the processing unit 2 performs image reconstruction by a confocal image reconstruction process to obtain a three-dimensional image from the calibrated signals. Fundamentally, the confocal image reconstruction process consists of coherently integrating the energy of each reflected signal at each focal point.

A two-dimensional imaging domain of the human head, as represented in FIG. 4, is considered. An antenna array where the antennas are placed equidistant from each other around the head is used. The positions of each of the antennas, represented in spherical coordinates (in, r) corresponding to Cartesian coordinates given by an=[xn, yn], where n is the number of the n-th antenna. The imaging area inside the head is represented by I, where the imaging points (focal points) inside are designated by im=[xm, ym], where m is the number of the m-th point in the imaging area.

To ensure coherent signal integration, the delay effects between the focal points and the antenna positions must be compensated. In this case, it is necessary to find the phase shift between each antenna and the other antennas. This delay time is equal to the direct distance between the transmitter and the receiver divided by the wave speed in the propagation medium. The next step is to extract the target location from the reflected signals. For this, all values are set to zero before their calculated delay. The signal propagation delay from the n-th antenna of the array to the m-th point of the imaging area, I, is calculated based on the following equation:

$${τ}_{n} ( {i}_{m} ) = \{ \frac{\sqrt{{( {x}_{n} - {x}_{m} )}^{2} + {( {y}_{n} - {y}_{m} )}^{2} \sqrt{{ε}_{e f f}}}}{c} \} \qquad [ Math . 2 ]$$

where ε_{eff} is the effective dielectric constant of the head. For the studied model, the value of ε_{eff} is calculated to be 38, which corresponds to the average of the measured dielectric constant values on artificial brains. To reconstruct an image, a focused beamforming algorithm, such as the delay-and-sum (DAS) beamforming algorithm, is implemented. For this, the first step is to identify the focal points to calculate the reflected signal energy model at these points, which, for the multi-static imaging algorithm, will be done by coherent integration of the signals. A delay-and-sum (DAS) beamforming algorithm creates a coherent integration of the energy of the signals at each focal point, by summing the phase-corrected signals relative to each other, according to the following equation:

$$I ( {i}_{m} ) = {\vert {\sum }_{n} {A}_{n} ( 2 ( {τ}_{n} ( {i}_{n} ) ) ) \vert}^{4} \qquad [ Math . 3 ]$$

where An is the signal of the antenna at location n (focal point of n).

The person skilled in the art knows how to switch from Cartesian coordinates to spherical coordinates.

FIGS. 5A-5D illustrate the confocal image reconstruction process. FIG. 5A shows the focal points inside the hemisphere with the size of the antenna distances.

The spatial precision between these points is about 3 mm. Then, by calculating the delay, these points are calculated from each antenna pair corresponding to each signal, and the energy accumulation at each point is obtained. FIG. 5B shows a three-dimensional diagram of a confocal image with DMAS beamforming. FIG. 5C shows a 2D image in coronal view, and FIG. 5D finally shows a two-dimensional image containing the target.

A combined method based on machine learning has been proposed for the segmentation and classification of CT/MRI images. The main steps of the proposed method are presented below.

A noise suppression method can be used to reduce the noise level in the images.

In addition, to reduce the dimensions of the images, dimension reduction methods, such as wavelet transform and principal component analysis (PCA), can be used. Finally, K-means methods, fuzzy image segmentation method, and multiclass SVM (support vector machine) segmentation method can be used to classify types of anomalies (strokes).

After creating the global three-dimensional image based on microwave imaging thanks to the availability of the MRI/CT image of the patient, these two images are combined in the post-processing program in the processing unit 2 and, after extracting the necessary information, a decision is made for feedback on the switching network to determine the parameters of the next scan. As this is a feedback loop method, the image must be displayed in several time frames.

To achieve the ultra-fast processing technique, signal reduction and sampling reduction are applied at two levels.

The main question is which antennas should be selected to have the maximum information capacity. The elements of the diffusion tensor show all possible connections, and the goal is to select the necessary signals based on the connectome from the diffusion tensor. In the context of cognitive scanning, it is not necessary to enter all the elements of the diffusion tensor into the image reconstruction algorithm. For example, among the different signals that pass through the same propagation channel, only one is selected and the others are eliminated to obtain a sparse matrix. This means that the lower triangular elements of the sparse matrix must be removed. In addition, due to the quasi-elliptical symmetry of the human head, the return signals from the antennas facing each other can be used for in-situ calibration. It should be noted that the return signals from each port are significantly different from the diagonal transmission signals, namely that the diagonal bi-static transmission signals are much weaker than the return signals in each monostatic port. This phenomenon is of considerable importance, as it governs the main imaging measures, such as correlation and contiguity of coded information from the scene when different channels are scanned.

A quantitative way to analyze the information capacity (and thus the orthogonality of spatial-temporal resolution) of the selected signals is to analyze the signal-to-clutter ratio (SCR) and the signal-to-mean ratio (SMR) of the reconstructed images and the target location in each scan. According to the invention, an SCR map and an SMR map are used as a means of detection and the target, in steps b and f. Then, based on the maximum value of these criteria (SCR and SMR), the position of the target is revealed. After creating the image by the confocal image reconstruction process during the first complete scan, information about the target position and other parameters, such as SCR, SMR are extracted from the current scan image. In addition, the intercorrelation matrix of the diffusion tensor must be compared to the SCR and SMR maps. All this information is then adapted to determine the position of the target and the area occupied by the target and is presented with respect to the connectome as a decision factor to determine the optimal selection of signals. Then, by comparing this position and the intercorrelation matrix, the signals that have the main effect on the target position are selected by examining the connectome connection and a compressive detection, implemented by the processing unit 2, is applied to these selected signals for the generation of sparse signals. In this case, the next scanned image is made using the fastest possible mode and the fewest necessary samples while preserving the required information related to the detected target. Compressive sampling is advantageously implemented by a convex optimization method of the L1 space.

After extracting information from the brain scan images of the patient, at this stage, using the image obtained by the microwave imaging method, new useful information is extracted to match the target location with the information extracted from the scan images and provide the necessary information in the feedback phase to decide on the second scan.

Due to the nature of microwave images, which are obtained by accumulating energy in focal points and which have target-like points in the image or blurred targets, target position detection uses data-based metrics. Useful metrics derived from this point of view comprise SCR and SMR. The region with the highest value of these two metrics is identified as the target region and compared to the target region obtained in the scan image, and as long as this comparison has the same response, instead of the entire signals of the diffusion tensor, selective signals are used for the next scan.

Due to the nature of microwave images, which are obtained by accumulating energy in focal points that have target-like points in the image or blurred targets, target position detection with artificial intelligence methods has a high error. Thus, to detect the target and extract its position from the image, techniques based on quantitative metrics are proposed. Useful metrics derived from this point of view are SCR and SMR. For this purpose, the image region has been divided into several 2D windows as shown in FIG. 6. The central location of the windows that has the highest value of these two metrics is identified as the target region.

The first parameter calculated in this invention is the SCR, which is used to evaluate how much the energy of the area is higher than the energy of the clutter in each 2D window S. Therefore, the SCR value quantifies the presence of an artifact at the target location in the brain. The SCR can be identified as follows:

$$SCR = \frac{{[ F ( n ) ]}_{S t r o k e}}{{[ F ( n ) ]}_{C l u t t e r}} \begin{matrix}∀ n ∈ S \\ ∀ n ∈ S\end{matrix} \qquad [ Math . 4 ]$$

where [F(n)]ₛₜᵣₒₖₑ is the energy value in the 2D window S in the presence of a target, and [F(n)]_{clutter} is the energy value in the same region S when the target is not present. Clutter is due to residual artifacts, and the average energy value of clutter is calculated in a background model, which is based on a healthy head model without stroke.

The second parameter calculated within the context of this invention is the SMR, which allows how much higher the energy of the target zone is than the average energy of the clutter in the head area to be evaluated. The SMR metric is specified as the ratio between the average value of the backscattered energy in the target area and the average value in the entire brain. The SMR can be identified as follows:

$$SMR = \frac{{[ F ( n ) ]}_{Stroke}}{{mean [ F ( n ) ]}_{Clutter}} \begin{matrix}∀ n ∈ S \\ ∀ n ∈ H\end{matrix} \qquad [ Math . 5 ]$$

where [F(n)]ₛₜᵣₒₖₑ is the intensity of all points inside the window S and mean [F(n)]_{clutter} is the intensity of all points inside the window H, where H represents the entire head region.

FIG. 7 shows the results of generating SCR metric maps from a 2D microwave image of FIG. 6, where the SCR value on the ordinate varies from 0 to 5.

We now describe the ultra-fast processing technique for a super-resolution microwave brain imaging system based on maximizing the extracted information capacity. After locating the target, the signals related to the target area are selected. Depending on the target position, relevant signals are selected in the target area to apply adaptive calibration. The adaptive connectome calibration process can be used for calibration. The hemispherical area of the helmet 4 can be divided into horizontal or vertical planes, each plane corresponding subset of the antenna array 5. The horizontal arrangement of signal selection can be applied depending on the height of the target area. In other words, if the detected target is at the same level as one of the circular arrangements of the antennas, only the signals related to this circular arrangement will be used to create the image in subsequent scans. Another arrangement in the vertical direction is possible, which, due to the lack of antennas, can only be effective in helping with better calibration.

The device 1 of the invention therefore performs a first scan of the brain of the patient using the helmet 4 carrying the antennas 5, by microwave imaging. A diffusion tensor is obtained, which is processed by the processing unit 2, which processes the obtained image by an SCR and SMR detection method to detect and position the detected target in the brain. Once the target is detected, a three-dimensional image is constructed by the adaptive connectome calibration process and compressive sampling, allowing a subgroup of the antenna array 5 to be selected, which will be most suitable for making a new image of the brain limited to the target region. Once the image limited to the target is constructed, also by the adaptive connectome calibration process and compressive sampling, this limited image is merged with the previous image or even with a more precise image obtained by fMRI or others, then the steps are thus repeated to obtain a sequence of images allowing the evolution of the target to be tracked, without having to recalculate the entire image each time. The power of the device according to the invention lies in the fact that the target is detected in each global image by a low-cost algorithm in calculation, the image capture being then limited to the region of the detected target to obtain a global image resulting from the merging of a more precise image with the image limited to the imaged area.

1. A method for three-dimensional brain imaging by microwave imaging, the method comprising the following steps:
a—performing, on a patient on whose head is placed a microwave antenna array controlled by a switching network connected to a controllable signal transceiver, a global three-dimensional image of the brain by microwave imaging using all the antennas of the microwave antenna array;
b—detecting at least one target in the global brain image made in step a;
c—calculating a position of the at least one target in the global brain image made in step a;
d—performing a local three-dimensional processing image of the target by microwave imaging using at least one among a subgroup of the microwave antenna array controlled by the switching network associated with the position of the at least one target detected in step a and a reduction in the number of samples made by the antennas;
e—merging the global image and the local processing image into a global intermediate three-dimensional image;
f—detecting at least one target in the global intermediate image;
g—calculating a position of the at least one target in the global intermediate image;
h—performing a new local three-dimensional processing image of the target by microwave imaging using at least one among a subgroup of the microwave antenna array controlled by the switching network associated with the position of the at least one target detected in step f and a reduction in the number of samples made by the antennas;
i—merging the global intermediate image and the new local processing image into a new global three-dimensional image also called intermediate image;
j—repeating steps f to i a predetermined number of times,
the at least one target being detected by calculating the signal-to-clutter ratio and the signal-to-mean ratio in two-dimensional planes of the three-dimensional image, the at least one target being detected on the surfaces of each two-dimensional plane on which the values of the signal-to-clutter ratio and the signal-to-mean ratio are simultaneously maximal.

2. The method according to claim 1, wherein each step of performing a three-dimensional image by microwave imaging comprises collecting diffusion parameter data representing microwaves scattered by the brain of the patient in a diffusion tensor, generating differential diffusion parameter data by an adaptive connectome calibration process to suppress clutter, and processing the differential diffusion parameter data by a confocal image reconstruction process to obtain a three-dimensional image.

3. The method according to claim 1, wherein the position of the at least one target in the three-dimensional image is calculated as the position of the surfaces of each two-dimensional plane on which the values of the signal-to-clutter ratio and the signal-to-mean ratio are simultaneously maximal.

4. The method according to claim 1, wherein the subgroup of the microwave antenna array associated with the position of the at least one target is constituted by the antennas having the shortest distance to the position of the at least one target.

5. The method according to claim 1, wherein compressive sampling is applied to the microwave signals transmitted by the antennas of the antenna array.

6. The method according to claim 1, wherein each step of merging two three-dimensional images consists of replacing, in the global image, the part corresponding to the local image, with the local image to obtain the merged image.

7. A microwave imaging device of the brain, comprising a helmet equipped with a microwave antenna array, a switching network controlling the microwave antenna array, a vector network analyzer connected to the switching network, a processing unit connected to the switching network and the vector network analyzer and controlling the switching network and the vector network analyzer, a human-machine interface connected to the processing unit, the processing unit comprising computing means and memory to perform the following steps:
a—performing a global three-dimensional image of the brain by microwave imaging using all the antennas of the microwave antenna array;
b—detecting at least one target in the global brain image made in step a;
c—calculating a position of the at least one target in the global brain image made in step a;
d—performing a local three-dimensional processing image of the target by microwave imaging using at least one among a subgroup of the microwave antenna array controlled by the switching network associated with the position of the at least one target detected in step a and a reduction in the number of samples made by the antennas;
e—merging the global image and the local processing image into a global intermediate three-dimensional image;
f—detecting at least one target in the global intermediate image;
g—calculating a position of the at least one target in the global intermediate image;
h—performing a new local three-dimensional processing image of the target by microwave imaging using at least one among a subgroup of the microwave antenna array controlled by the switching network associated with the position of the at least one target detected in step f and a reduction in the number of samples made by the antennas;
i—merging the global intermediate image and the new local processing image into a new global three-dimensional image also called intermediate image;
j—repeating steps f to i a predetermined number of times,
the at least one target being detected by calculating the signal-to-clutter ratio and the signal-to-mean ratio in two-dimensional planes of the three-dimensional image, the at least one target being detected on the surfaces of each two-dimensional plane on which the values of the signal-to-clutter ratio and the signal-to-mean ratio are simultaneously maximal.

8. The device according to claim 7, wherein each step of performing a three-dimensional image by microwave imaging comprises collecting diffusion parameter data representing microwaves scattered by the brain of the patient in a diffusion tensor, generating differential diffusion parameter data by an adaptive connectome calibration process to suppress clutter, and processing the differential diffusion parameter data by a confocal image reconstruction process to obtain a three-dimensional image.

9. The device according to claim 8, wherein the position of the at least one target in the three-dimensional image is calculated as the position of the surfaces of each two-dimensional plane on which the values of the signal-to-clutter ratio and the signal-to-mean ratio are simultaneously maximal.

10. The device according to claim 7, wherein the subgroup of the microwave antenna array associated with the position of the at least one target is constituted by the antennas-having the shortest distance to the position of the at least one target.

11. The device according to claim 7, wherein compressive sampling is applied to the microwave signals transmitted by the antennas of the antenna array.

12. The device according to claim 7, wherein each step of merging two three-dimensional images consists of replacing, in the global image, the part corresponding to the local image, with the local image to obtain the merged image.

13. The device of brain microwave imaging according to claim 7, wherein the antenna array comprises 24 antennas, preferably butterfly antennas.

14. A computer program product comprising instructions which, when executed by a microwave imaging device according to claim 7, perform the following steps:
a—performing a global three-dimensional image of the brain by microwave imaging using all the antennas of the microwave antenna array;
b—detecting at least one target in the global brain image made in step a;
c—calculating a position of the at least one target in the global brain image made in step a;
d—performing a local three-dimensional processing image of the target by microwave imaging using at least one among a subgroup of the microwave antenna array controlled by the switching network associated with the position of the at least one target detected in step a and a reduction in the number of samples made by the antennas;
e—merging the global image and the local processing image into a global intermediate three-dimensional image;
f—detecting at least one target in the global intermediate image;
g—calculating a position of the at least one target in the global intermediate image;
h—performing a new local three-dimensional processing image of the target by microwave imaging using at least one among a subgroup of the microwave antenna array controlled by the switching network associated with the position of the at least one target detected in step f and a reduction in the number of samples made by the antennas;
i—merging the global intermediate image and the new local processing image into a new global three-dimensional image also called intermediate image;
j—repeating steps f to i a predetermined number of times.
